(19) United States
(12) Patent Application Publication
(10) Pub. No.: US 2021/0330068 A1
(43) Pub. Date: Oct. 28, 2021

(54) ORAL ELECTRICAL CLEANING DEVICE

(71) Applicant: BZL Medical Ltd., Habonim (IL)

(72) Inventor: BenZion Levi, Habonim (IL)

(73) Assignee: BZL Medical Ltd., Habonim (IL)

(21) Appl. No.: 16/858,992

(22) Filed: Apr. 27, 2020

(51) Int. Cl. A46B 15/00; A46B 9/04; A46B 13/02; A46B 13/00; A61C 17/26; A61C 17/34; A61N 1/05

(52) U.S. Cl. CPC A46B 15/0022; A46B 9/04; A46B 13/023; A61N 1/322; A61C 17/26; A61C 17/3481; A61N 1/0548; A46B 13/008

(57) ABSTRACT

An oral cleaning device includes a head portion, RF electrodes located on the head portion and a non-conductive barrier located on the head portion that separates the electrodes from each other. The electrodes, bristles (if included) and the non-conductive barrier are mounted on a base and the base is arranged for either vibratory or rotational motion.

### FIELD OF THE INVENTION

The present invention relates generally to oral cleaning devices, and particularly to an oral cleaning device, such as a toothbrush, that generates a chemical agent in situ by applying RF energy to electrodes on the cleaning device in the presence of substances in the user's mouth.

### BACKGROUND OF THE INVENTION

U.S. patent Ser. No. 10/201,701 to Levi describes an oral cleaning device, such as a toothbrush, that generates a chemical agent in situ by applying RF energy and/or microcurrent source to a conductive surface and/or to electrodes on the cleaning device in the presence of substances in the user's mouth

### SUMMARY OF THE INVENTION

The present invention seeks to provide further improvements to the oral cleaning device of U.S. patent Ser. No. 10/201,701, as is described more in detail hereinbelow.

The oral cleaning device of the invention includes a RF (radio frequency) generating toothbrush head formed with electrodes and having a brush implanted in one end thereof; and an RF generating circuit connected to the electrodes for providing high frequency alternate voltage to the electrodes. A non-conductive barrier defines the path over which the high frequency alternate current flows between the electrodes.

The invention may be used for cleansing, bleaching, and sanitizing tooth or other surfaces.

### BRIEF DESCRIPTION OF THE DRAWINGS

The present invention will be understood and appreciated more fully from the following detailed description, taken in conjunction with the drawings in which:

FIG. 1 is a simplified front view illustration of a bristle head portion of an oral cleaning device, in accordance with an embodiment of the invention.

FIGS. 2 and 3 are simplified front view and side view illustrations, respectively, of a bristle head portion, in accordance with another embodiment of the invention, with two electrodes (one electrode that can be divided into several parts) and a non-conductive barrier between them.

FIGS. 4 and 5 are simplified front view and side view illustrations, respectively, of a bristle head portion, in accordance with another embodiment of the invention, with two electrodes (each electrode can be divided into several parts) and a non-conductive barrier between them.

FIG. 6 is a simplified illustration of vibrational movement of the bristles and non-conducted barrier.

FIG. 7 is a simplified illustration of rotational movement of the bristles and non-conducted barrier.

FIG. 8 is a simplified side view illustration of a bristle head portion of an oral cleaning device, in accordance with another embodiment of the invention, with a central, straight first electrode flush with the base and two straight peripheral second electrodes, with non-conducting barriers between the first and second electrodes.

FIG. 9 is a simplified side view illustration of a bristle head portion of an oral cleaning device, in accordance with another embodiment of the invention, with a central, straight first electrode protruding from the base and two straight peripheral second electrodes, with non-conducting barriers between the first and second electrodes.

FIG. 10 is a simplified front view illustration of the bristle head portions of FIGS. 8 and 9;

FIG. 11 is a simplified side view illustration of a bristle head portion of an oral cleaning device, in accordance with another embodiment of the invention, with first and second electrodes flush with the base and a non-conducting barrier (e.g., shaped from the same material of the base) between the first and second electrodes.

FIG. 12 is a simplified side view illustration of a bristle head portion of an oral cleaning device, in accordance with another embodiment of the invention, with first and second electrodes protruding from the base and a non-conducting barrier e.g., (shaped from the same material of the base) between the first and second electrodes.

FIG. 13 is a simplified front view illustration of the bristle head portions of FIGS. 11 and 12;

FIG. 14 is a simplified side view illustration of a bristle head portion of an oral cleaning device, in accordance with another embodiment of the invention, with first and second electrodes flush with a bristle base and a non-conducting barrier between the first and second electrodes.

FIG. 15 is a simplified side view illustration of a bristle head portion of an oral cleaning device, in accordance with another embodiment of the invention, with first and second electrodes protruding from the bristle base and a non-conducting barrier between the first and second electrodes.

FIG. 16 is a simplified front view illustration of the bristle head portions of FIGS. 14 and 15;

FIG. 17 is a simplified side view illustration of a bristle head portion of an oral cleaning device, in accordance with another embodiment of the invention, with first and second electrodes flush with the base and a non-conducting barrier between the first and second electrodes.

FIG. 18 is a simplified side view illustration of a bristle head portion of an oral cleaning device, in accordance with another embodiment of the invention, with first and second electrodes protruding from the base and a non-conducting barrier between the first and second electrodes.

FIG. 19 is a simplified front view illustration of the bristle head portions of FIGS. 17 and 18;

FIGS. 20 and 21 are simplified front view and side view illustrations, respectively, of a head portion, in accordance with another embodiment of the invention.

FIG. 22 is a simplified side view illustration of the head portions of FIGS. 20 and 21, with a dentifrice applied to the head portion.

### DETAILED DESCRIPTION OF EMBODIMENTS

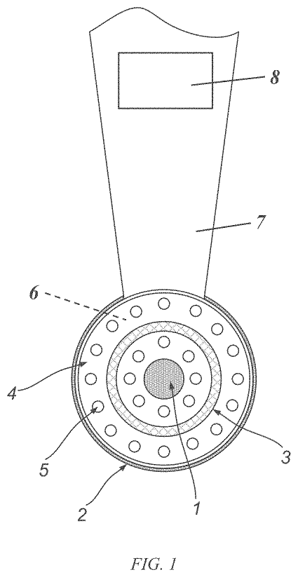

Reference is now made to FIG. 1, which illustrates a bristle head portion of an oral cleaning device, constructed and operative in accordance with a non-limiting embodiment of the present invention.

The bristle head portion of the oral cleaning device includes a first electrode 1 (which is the RF first pole), a second electrode 2 (which is the RF second pole), a non-conducting barrier 3, a base 4 and one more bristles 5 (also referred to as cleaning elements). Base 4 is arranged for either vibration or rotation, and may be coupled to an actuator 6 that provides this motion (alternatively the motion may be done manually). As base 4 vibrates in oscillation (FIG. 6) or rotation (FIG. 7), the non-conducting barrier 3 and bristles 5 also vibrate or rotate; however, the first and second electrodes 1 and 2 preferably do not rotate.

In FIG. 1, the first and second electrodes 1 and 2 are respectively a central electrode and a peripheral ring electrode. The ring electrode (second electrode 2) spans the complete periphery of the base 4. The non-conducting barrier 3 is a complete annular barrier. The device, as in all other embodiments, may include a handle portion 7, which may be grasped by the user or which may serve as the interface of an accessory to be inserted in a handle of an electric toothbrush or other oral care device.

In this embodiment, as in all other embodiments, an RF (radio frequency energy) generator 8 is provided (such as in handle portion 7 or external thereto), for generating RF energy in a frequency range, typically but not limited to, 500 KHz-30 MHz (such as 3 MHz).

In all embodiments, the electrodes serve as RF poles and make contact with an electrolyte within a user's mouth and generate chemical agents in situ at the application site of the agents. The electrolyte may include saliva alone, a dentifrice in the presence of saliva, and/or a mixture of saliva, dentifrice and conductivity agents, such as salts, which increase the conductivity of an aqueous solution.

The RF generator 8 is electrically connected to electrodes 1 and 2 for providing RF energy to the electrodes 1 and 2. The electrodes may operate in a bipolar mode and enhance the efficacy of the chemical agents in the user's mouth.

The device can generate the chemical agents in a variety of ways depending on factors such as the configuration of the toothbrush, the RF energy (such as but not limited to, frequency 3 MHz and 22 Vrms) provided to the electrodes, the composition of the electrolyte, the composition of the electrodes and other factors. For instance, in the presence of saliva alone, the device can generate hydrogen, oxygen, peroxide and ozone; in the presence of ionic compounds such as sodium chloride, potassium chloride, or calcium carbonate that may be contained in an aqueous dentifrice, chemical agents such as chlorine or calcium can be generated; in another example, in the presence of a dentifrice containing an activatable chemical agent that is stored in an inactive state, the chemical agent can be generated by being activated via the RF energy.

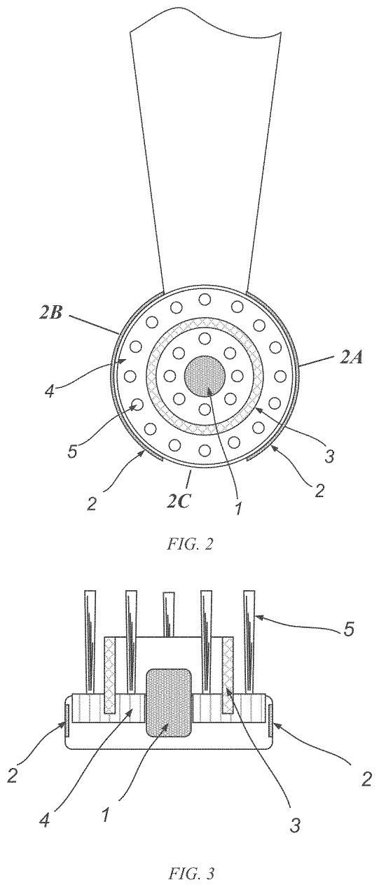

In FIGS. 2 and 3, the first and second electrodes 1 and 2 are respectively a central electrode and a peripheral ring electrode. The ring electrode (second electrode 2) does not span the complete periphery of the base 4; instead it has two partial annular portions 2A and 2B separated by a gap 2C. The non-conducting barrier 3 is a complete annular barrier.

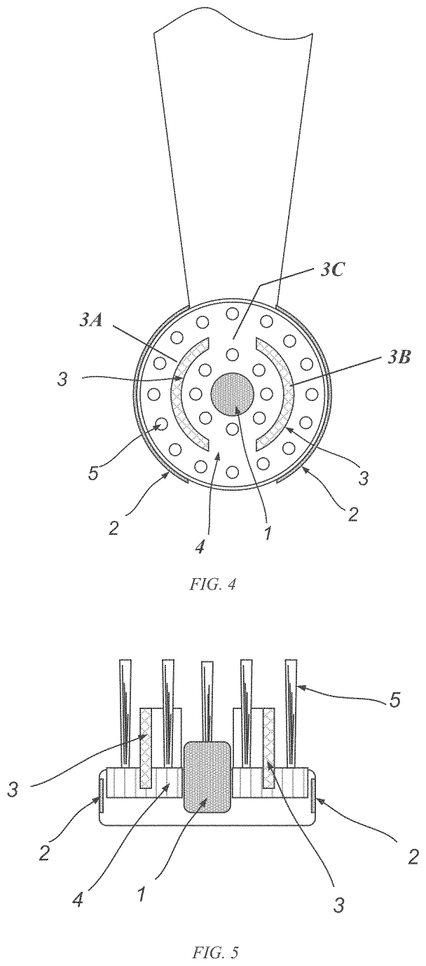

In FIGS. 4 and 5, the structure is the same as in FIGS. 2 and 3, except that the non-conducting barrier 3 has two partial annular portions 3A and 3B separated by one or more gaps 3C.

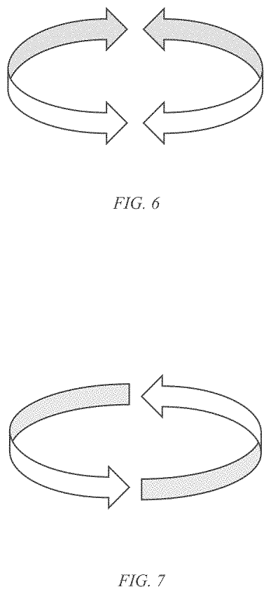

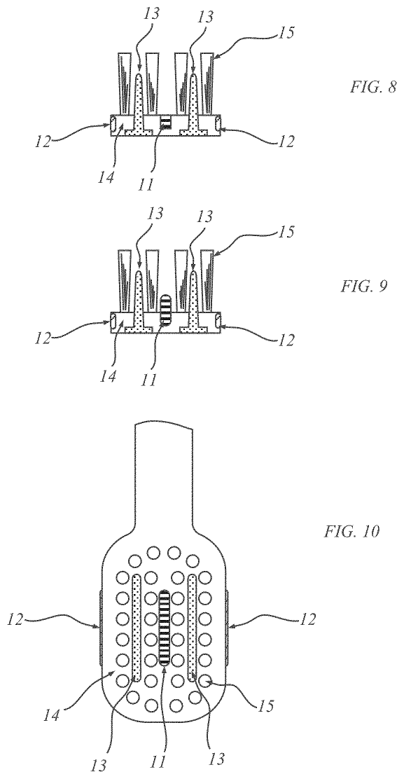

Reference is now made to FIG. 8, which illustrates a bristle head portion of an oral cleaning device, in accordance with another embodiment of the invention. In this embodiment, there is a central, straight first electrode 11 flush with a base 14 and two straight peripheral second electrodes 12, with non-conducting barriers 13 between the first and second electrodes 11 and 12. FIG. 9 illustrates a similar embodiment, except that the central, straight first electrode 11 protrudes from the base 14. One or more cleaning elements (bristles) 15 are distributed on base 14 between the electrodes and non-conductive barrier.

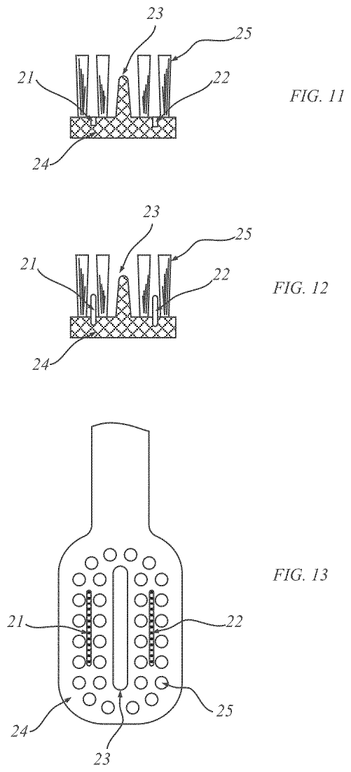

Reference is now made to FIG. 11, which illustrates a bristle head portion of an oral cleaning device, in accordance with another embodiment of the invention, with first and second electrodes 21 and 22 flush with a base 24 and a non-conducting barrier 23 (which may be part of the base 24) between the first and second electrodes 21 and 22. FIG. 12 illustrates a similar embodiment, except that the first and second electrodes 21 and 22 protrude from the base 24. One or more cleaning elements (bristles) 25 are distributed on base 24 between the electrodes and non-conductive barrier.

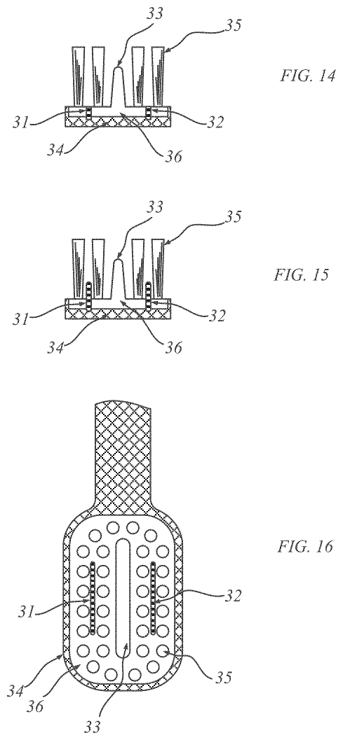

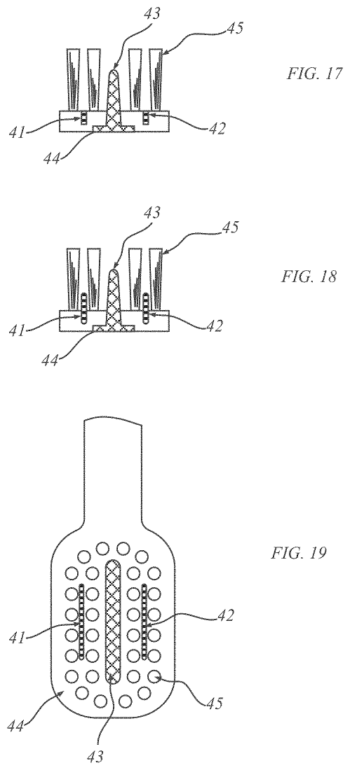

Reference is now made to FIG. 14, which illustrates a bristle head portion of an oral cleaning device, in accordance with another embodiment of the invention, with first and second electrodes 31 and 32 flush with a bristle base 36 (mounted on a base 34) and a non-conducting barrier 33 between the first and second electrodes 31 and 32. The non-conducting barrier 33 is part of the bristle base 36. FIG. 15 illustrates a similar embodiment, except that the first and second electrodes 31 and 32 protrude from the bristle base 36. One or more cleaning elements (bristles) 35 are distributed on bristle base 36 between the electrodes and non-conductive barrier.

Reference is now made to FIG. 17, which illustrates a bristle head portion of an oral cleaning device, in accordance with another embodiment of the invention, with first and second electrodes 41 and 42 flush with a base 44. A non-conducting barrier 43, which is embedded down to a bottom face of base 44, is located between the first and second electrodes 41 and 42. FIG. 18 illustrates a similar embodiment, except that the first and second electrodes 41 and 42 protrude from the base 44. One or more cleaning elements (bristles) 45 are distributed on base 44 between the electrodes and non-conductive barrier.

The different heights of the electrodes in the illustrated embodiments create different electrical and electromagnetic fields in the vicinity of the brush head and barrier.

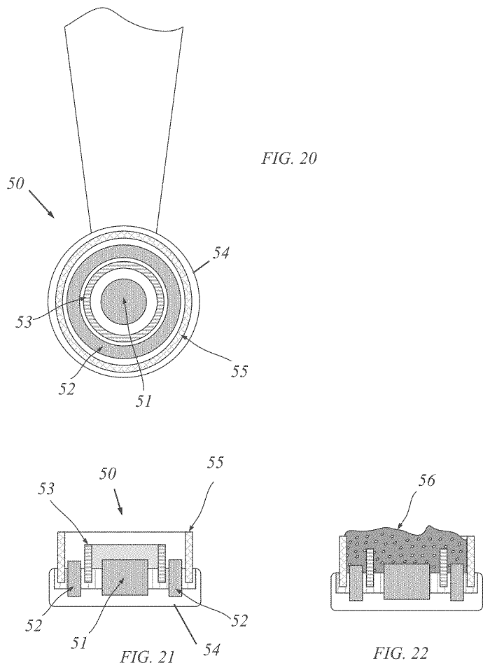

Reference is now made to FIGS. 20 and 21, which illustrate a head portion 50, in accordance with another embodiment of the invention. Head portion 50 does not have bristles. Head portion 50 includes first and second electrodes 51 and 52, which are respectively a central electrode and an outer ring electrode which protrude from (or are flush with) a base 54. The ring electrode (second electrode 52) is a complete ring, but may be partial rings separated by gaps as in previous embodiments. An inner non-conducting barrier 53 is a complete annular barrier between first and second electrodes 51 and 52, but may include partial rings separated by gaps as in previous embodiments. An outer non-conducting barrier 55 is located outside of second electrode 52.

As seen in FIG. 22, a dentifrice 56 may be applied to the head portion 50. The dentifrice is applied over the electrodes and inner non-conducting barrier, and extends to the outer non-conducting barrier. There may be more than one such containment area for the dentifrice or for different dentifrices. As with the other embodiments, here, too, the head portion may be vibratory or rotational.

What is claimed is:

1. An oral cleaning device comprising:
a head portion;
RF (radio frequency) electrodes located on said head portion;
a non-conductive barrier located on said head portion that separates said electrodes from each other; and
wherein said electrodes and said non-conductive barrier are mounted on a base and said base is arranged for either vibratory or rotational motion.

2. The oral cleaning device according to claim 1, wherein said head portion comprises cleaning elements.

3. The oral cleaning device according to claim 1, wherein at least one of said electrodes and said non-conductive barrier is an at least partial ring.

4. An oral cleaning device comprising:
a head portion;
RF electrodes located on said head portion;
a non-conductive barrier located on said head portion that separates said electrodes from each other; and
wherein at least one of said electrodes and said non-conductive barrier is an at least partial ring.

5. The oral cleaning device according to claim 3, wherein said head portion comprises cleaning elements.
